(19) United States
(12) Patent Application Publication
(10) Pub. No.: US 2025/0090616 A1
(43) Pub. Date: Mar. 20, 2025

(54) COMPOSITION FOR IMPROVING SLEEP

(71) Applicant: Ruchir P. Patel, Scottsdale, AZ (US)

(72) Inventor: Ruchir P. Patel, Scottsdale, AZ (US)

(21) Appl. No.: 18/369,717

(22) Filed: Sep. 18, 2023

(51) Int. Cl. A61K 36/736; A61K 31/00; A61K 36/24; A61P 25/20

(52) U.S. Cl. CPC A61K 36/736; A61K 31/658; A61K 36/24; A61P 25/20

(57) ABSTRACT

A natural and organic composition for improving sleep problems and insomnia in human adults. The composition includes between 100 mg to 3000 mg of tart cherry (Prunus cerasus) extract and between 25 mg to 100 mg of Apocynum venetum leaf extract. The composition may also include between 10 mg to 100 mg of cannabidiol. The composition helps to improve sleep quality, daytime awareness, and sleep duration, while also decreasing sleep latency, insomnia severity scores, and generalized anxiety scores.

### TECHNICAL FIELD

This disclosure generally relates to sleep aids and, more particularly, to a composition that may be used to help improve sleep problems and insomnia in human adults.

### BACKGROUND OF THE INVENTION

Many people experience some form of sleep disturbance. The most common sleep disturbance is insomnia, which is when an individual has difficulty falling asleep and staying asleep. Individuals may also experience other sleep problems relating to sleep duration and sleep latency. These sleep problems and sleep disturbances may ultimately affect the individual's anxiety and alertness levels during the daytime.

Currently, there are medications available to treat sleep disturbances. These medications include triazolam, estazolam, temazepam, quazepam,, zolpidem, eszoplicone, zaleplon, diphenhydramine, trazodone, lemborexant, suvorexant, daridorexant, and flurazepam. However, these medications may be harmful for individuals because of their negative side effects and potential for abuse or dependance, as well as increased risk of developing cognitive dysfunction. Therefore, a need exists for a natural and organic composition for improving sleep problems and insomnia in human adults.

### SUMMARY OF THE INVENTION

This summary is provided to introduce a selection of concepts in a simplified form that are further described below in the DETAILED DESCRIPTION OF THE INVENTION. This summary is not intended to identify key features of the claimed subject matter, nor is it intended to be used as an aid in determining the scope of the claimed subject matter.

In accordance with one embodiment of the present invention, a composition for improving sleep is disclosed. The composition comprises: an effective amount of tart cherry extract; and an effective amount of Apocynum venetum leaf extract.

In accordance with another embodiment of the present invention, a composition for improving sleep is disclosed. The composition comprises: between 100 mg to 3000 mg of Prunus cerasus extract; and between 25 mg to 100 mg of Apocynum venetum leaf extract.

In accordance with another embodiment of the present invention, a liquid composition for improving sleep is disclosed. The liquid composition comprises: 553 mg of Prunus cerasus extract; 25 mg of Apocynum venetum leaf extract; and between 10 mg to 100 mg of cannabidiol.

### DETAILED DESCRIPTION OF THE INVENTION

The description set forth below is intended as a description of presently preferred embodiments of the disclosure and is not intended to represent the only forms in which the present disclosure can be constructed and/or utilized. The description sets forth the functions and the sequence of steps for constructing and operating the disclosure. It is to be understood, however, that the same or equivalent functions and sequences can be accomplished by different embodiments that are also intended to be encompassed within the spirit and scope of this disclosure.

The all-natural, organic composition of the present invention may be used to help improve sleep problems and insomnia experienced by adult humans. It was developed to help improve sleep problems and insomnia by identifying the root cause(s) of the issues. In its simplest form, the composition contains an effective amount of tart cherry extract and an effective amount of extract derived from the Rafuma leaf, Apocynum venetum.

Tart cherries, Prunus cerasus, are also known as Montmorency cherries, sour cherries, or dwarf cherries. Montmorency cherries are known to provide a natural source of melatonin, which is a hormone that regulates the sleep-wake cycle in humans. Proanthocyanidins, the chemical compounds that give the tart cherry its color, help to increase the brain availability of tryptophan. Tryptophan is an essential amino acid that is converted into serotonin and serotonin helps control a human's mood and sleep. The minimization of tryptophan degradation may allow tryptophan to work more effectively in the body while increasing bioavailability for serotonin synthesis, thus leading to a positive effect on sleep, improving mood and decreasing inflammation.

Apocynum venetum leaf extract has been known to have beneficial effects on sleep. VENETRON® extract is dietary supplement that contains Apocynum venetum leaf extract. VENETRON® extract is a purified, powdered extract derived from the Rafuma leaf, Apocynum venetum. It is known to contain bioactive flavonoids and has been found to reduce the degradation of serotonin in the blood and brain. VENETRON® extract has been used to produce a calming effect in a user by acting on the GABA-ergic system.

Together, Montmorency tart cherry extract and VENETRON® extract may be used as a powerful, natural solution to insomnia. They work to increase the presence of serotonin in the brain to improve sleep. Additionally, together they improve mood of a human because VENETRON® extract exerts a calming effect on the mind and helps the overactive mind relax for a quicker, longer-lasting sleep.

### EXAMPLES

A liquid composition in accordance with the present invention was prepared according to the formulation shown in Table 1 below and was used in the following examples where one serving size is equal to ¼ teaspoon (1.23 ml):

TABLE 1
Composition

| Component | Amount |
|---|---|
| Tart cherry (Prunus cerasus) | 553 mg |
| Apocynum venetum leaf extract | 25 mg |

An observational study with the purpose of examining the effectiveness of the composition of the present invention on sleep quality was conducted for participants with self-reported sleep disturbance. Specifically, the participants in the study experienced moderate to severe insomnia based on an Insomnia Severity Index (ISI) score≥15. The composition shown in Table 1 was used in the observational study. The study also explored any changes on symptoms of sleep disturbance, on anxiety, on the level of alertness during daytime, and on sleep duration and sleep latency using data obtained from participants' personal wearable devices. The wearable devices were electronic devices worn on the users' bodies and included smartwatches and fitness trackers.

The study was done over a period of four weeks, consisting of two weeks without the use of the composition of the present invention and two weeks total using the composition; i.e. one week off (baseline)—one week on—one week off—and one week on for each participant. During the weeks using the composition (weeks on), participants were asked to take a nightly dose of the composition 30-60 minutes prior to laying down in bed. The composition was administered orally by the participant placing ¼ teaspoon of the composition under the participant's tongue and then swallowing.

During the four-week study duration, participants completed a daily survey that asked about sleep quality, the time they laid in bed the night before, and how rested they felt since waking (daytime alertness). Objective data for total sleep duration and bed time to calculate for sleep latency were collected from participants' personal wearable devices. Participants also completed the Insomnia Severity Index (ISI) and Generalized Anxiety Disorder-7 Assessment (GAD-7) during baseline, at the beginning of each week and at the end of the study.

One hundred twenty (120) participants aged 18+ consented and were screened for the study. Thirty-three (33) participants failed screening and ten (10) participants opted out or decided not to continue.

Seventy-seven (77) participants were enrolled and received the composition of the present invention. Three (3) participants withdrew from the study and twelve (12) participants were lost to follow-up. Sixty-two (62) participants completed 4 weeks of the study with 98.1% data compliance.

In order to be included in the data analysis, participants had to have completed at least 60% of the daily assessments in each arm (Composition Use vs. No Composition Use) and were required to have objective sleep data collected from their personal wearable devices. Forty-three (43) participants met the above criteria and were considered evaluable.

The demographics of the 43 evaluable participants were as follows:
a. Age: 18-29 (2%); 30-39 (21%); 40-49 (16%); 50-59 (45%); 60-69 (9%); 70-79 (2%); and >80 (5%)
b. Race/Ethnicity: White/Caucasian (86%); Black/African American (7%); Asian/Asian American (2%); and LatinX or Hispanic (5%)
c. Sex: Women (76.7%) and Men (23.3%)

### Example 1: Sleep Quality

Participants answered a daily question for four weeks in which they rated their overall sleep quality from the night before on a scale from 0-10. The results were recorded in the Table 2 below.

TABLE 2
Median Values of Daily Sleep Quality Scores

| | Off | On | | |
|---|---|---|---|---|
| | Composition | Composition | | |
| | (median) | (median) | Difference | P value |
| Week 1 vs 2 | 4.4 | 5.4 | 1.0 | <0.001 |
| Week 3 vs 2 | 5.2 | 5.4 | 0.2 | 0.02 |
| Week 3 vs 4 | 5.2 | 6.1 | 0.9 | <0.001 |
| Week 1 vs 4 | 4.4 | 6.1 | 1.7 | <0.001 |
| Weeks 1&3 vs 2&4 | 4.7 | 5.8 | 1.1 | <0.001 |

Results from the study report the proportion of participants with a statistically significant increase and any increase in sleep quality. Individual's statistically significant change from no composition use to composition use is defined as a p-value<0.05 under the Mann-Whitney U Test.

The results show that 15 (or 35%) of the participants showed a significant increase in sleep quality and also showed that 36 (or 84%) of the participants showed any increase in sleep quality. The average sleep quality change for all participants was an increased 0.82±0.74 points. The average sleep quality change for participants with any increase was an increased 1.02±0.61 points. The average sleep quality change for participants with significant increase was an increased 1.38 0.61 points. Therefore, there is a statistically significant increase in sleep quality in the weeks that the composition was used versus the weeks without the composition. Analysis of gender distribution showed no significant difference between male and female participants with increase in sleep quality.

### Example 2: Daytime Alertness

Participants answered a daily question for four weeks in which they rated how rested they felt since waking on a scale from 0-10. The results were recorded in the Table 3 below.

TABLE 3
Median Values of Daily Daytime Alertness Scores

| | Off | On | | |
|---|---|---|---|---|
| | Composition | Composition | | |
| | (median) | (median) | Difference | P value |
| Week 1 vs 2 | 5 | 5 | 0 | 0.02 |
| Week 3 vs 2 | 4 | 5 | 1 | 0.05 |
| Week 3 vs 4 | 4 | 6 | 2 | 0.01 |
| Week 1 vs 4 | 5 | 6 | 1 | 0.07 |
| Weeks 1&3 vs 2&4 | 5 | 6 | 1 | <0.001 |

Results from the study report the proportion of participants with a statistically significant increase and any increase in daytime alertness. Individual's statistically significant change from no composition use to composition use is defined as a p-value<0.05 under the Mann-Whitney U Test.

The results also show that 12 (or 28%) of the participants showed a significant increase in daytime alertness and also showed that 38 (or 88%) of the participants showed any increase in daytime alertness. The average change in daytime alertness for all participants was an increased 0.79±0.70 points. The average change in daytime alertness for participants with any increase was an increased 0.93±0.60 points. The average change in daytime alertness for participants with significant increase was an increased 1.47±0.59 points. Therefore, there is a statistically significant increase in daytime alertness in the weeks that the composition was used versus the weeks without the composition. Analysis of gender distribution showed no significant difference between male and female participants with increase in daytime alertness.

### Example 3: Sleep Duration

Daily sleep duration was obtained from participants' personal wearable devices over four weeks. The results were recorded in the Table 4 below.

TABLE 4
Median Values of Daily Sleep Duration

| | Off | On | | |
|---|---|---|---|---|
| | Composition | Composition | | |
| | (median) | (median) | Difference | P value |
| Week 1 vs 2 | 6.8 | 6.6 | −0.2 | 0.17 |
| Week 3 vs 2 | 6.8 | 6.6 | 0.2 | 0.85 |
| Week 3 vs 4 | 6.8 | 6.8 | 0 | 0.81 |
| Week 1 vs 4 | 6.8 | 6.8 | 0 | 0.63 |
| Weeks 1&3 vs 2&4 | 6.9 | 6.8 | −0.1 | 0.31 |

Results from the study indicate that there was no detectable significant change in sleep duration between the weeks that the composition was used versus the weeks without the composition. Individual's statistically significant change from no composition use to composition use is defined as a p-value<0.05 under the Mann-Whitney U Test.

The results also show that one (or 2%) of the participants showed a significant increase in sleep duration and also showed that 21 (or 49%) of the participants showed any increase in sleep duration. The average sleep duration change for all participants was a decreased 10.8±63.6 minutes. The average sleep duration change for participants with any increase was an increased 35.4±31.2 minutes.

### Example 4: Daily Sleep Latency

Daily sleep latency was calculated from the participants' manually entered time that they laid in bed and bed time obtained from participants' personal wearable device over four weeks. The results were recorded in the Table 5 below.

TABLE 5
Median Values of Daily Sleep Latency

| | Off | On | | |
|---|---|---|---|---|
| | Composition | Composition | | |
| | (median) | (median) | Difference | P value |
| Week 1 vs 2 | 39.7 | 24.6 | 15.1 | 0.08 |
| Week 3 vs 2 | 34.4 | 24.6 | −9.8 | 0.22 |
| Week 3 vs 4 | 34.4 | 39.2 | −4.8 | 0.43 |
| Week 1 vs 4 | 39.7 | 39.2 | 0.5 | 0.46 |
| Weeks 1&3 vs 2&4 | 37.3 | 38.5 | −1.2 | 0.76 |

Results from the study indicate that there was no detectable significant change in sleep latency between the weeks that the composition was used versus the weeks without the composition. 11 participants were excluded due to insufficient data to calculate individual statistics. Individual's statistically significant change from no composition use to composition use is defined as a p-value<0.05 under the Mann-Whitney U Test.

The results show that none of the participants showed a significant decrease in sleep latency and also showed that 18 (or 56%) of the participants showed any decrease in sleep latency. The average sleep latency change for all participants showed an increased 0.09±17.9 minutes. The average sleep latency change for participants with any decrease in sleep latency showed a decreased 11.1±8.12 minutes.

### Example 5: Insomnia Severity Index (ISI)

The Insomnia Severity Index is a questionnaire that assesses the nature, severity, and impact of insomnia in adults. It consists of seven (7) questions that participants score on a scale of 0-4. Each question's score is added together for the final ISI score. The ISI was collected four times during the study period at the start of each week. The ISI questions were as indicated in Table 6 below and the ISI Scoring Interpretations were as indicated in Table 7 below.

TABLE 6
ISI Questions

| | |
|---|---|
| Question 1 | Difficulty falling asleep? |
| Question 2 | Difficulty staying asleep? |
| Question 3 | Problem waking up too early? |
| Question 4 | How SATISFIED/DISSATISFIED are you with your |
| | CURRENT sleep pattern? |
| Question 5 | How NOTICEABLE to others do you think your sleep |
| | problem is in terms of impairing the quality of your life? |
| Question 6 | How WORRIED/DISTRESSED are you about your current |
| | sleep problem? |
| Question 7 | To what extent do you consider your sleep problem to |
| | INTERFERE with your daily functioning (e.g. daytime |
| | fatigue, mood, ability to function at work/daily chores, |
| | concentration, memory, mood, etc.) CURRENTLY? |

TABLE 7
ISI Scoring Interpretation

| Total Score | Interpretation |
|---|---|
| 0-7 | No clinically significant insomnia |
| 8-14 | Subthreshold insomnia |
| 15-21 | Clinical insomnia (moderate severity) |
| 22-28 | Clinical insomnia (severe) |

The results from the study were recorded in the Table 8 below.

TABLE 8
Medial Values of Weekly ISI Scores

| | Off | On | | |
|---|---|---|---|---|
| | Composition | Composition | | |
| | (median) | (median) | Difference | P value |
| Week 1 vs 2 | 20 | 15 | 5 | <0.001 |
| Week 3 vs 2 | 15 | 15 | 0 | 0.11 |
| Week 3 vs 4 | 15 | 12 | 3 | <0.001 |
| Week 1 vs 4 | 20 | 12 | 8 | <0.001 |
| Weeks 1&3 vs 2&4 | 17 | 13 | 4 | <0.001 |

Results from the study indicate that there was a statistically significant decrease in the ISI score between the weeks that the composition was used versus the weeks without the composition. Individual's statistically significant change from no composition use to composition use is defined as a p-value<0.05 under the Mann-Whitney U Test.

The results show that 41 (or 95%) of participants showed a decrease in ISI score. The average ISI score when the participant was not using the composition was 17.4±3.64. The average ISI score when the participant was using the composition was 13.4±4.23.

### Example 6: Generalized Anxiety Disorder-7 Assessment (GAD-7)

The Generalized Anxiety Disorder-7 is an easy-to use self-administered patient questionnaire for identifying and measuring the severity measure of generalized anxiety disorder (GAD). It consists of seven (7) questions that participants score on a scale of 0-3. Each question's score is added together for the final GAD-7 score. The GAD-7 was collected four times during the study period at the start of each week.

The results from the study were recorded in the Table 9 below.

TABLE 9
Median Values of Weekly GAD-7 Scores

| | Off | On | | |
|---|---|---|---|---|
| | Composition | Composition | | |
| | (median) | (median) | Difference | P value |
| Week 1 vs 2 | 7 | 4 | 3 | <0.001 |
| Week 3 vs 2 | 4 | 4 | 0 | 0.50 |
| Week 3 vs 4 | 4 | 3 | 1 | 0.001 |
| Week 1 vs 4 | 7 | 3 | 4 | 0.08 |
| Weeks 1&3 vs 2&4 | 7 | 4 | 3 | <0.001 |

Results from the study indicate that there was a statistically significant decrease in the GAD-7 score with the use of the composition versus without the use of the composition. Individual's statistically significant change from no composition use to composition use is defined as a p-value<0.05 under the Mann-Whitney U Test.

The results show that 34 (or 79%) of participants showed a decrease in GAD-7 score. The average GAD-7 score when the participant was not using the composition was 8.3±4.91. The average GAD-7 score when the participant was on the composition was 5.88±3.64.

In the examples above, one serving (1.23 ml) of the composition comprised 553 mg sour cherry (Prunus cerasus) 1:5 fruit extract and 25 mg Apocynum venetum leaf extract. However, it should be clearly understood that substantial benefit may also be derived from a composition containing other amounts of sour cherry (Prunus cerasus) and Apocynum venetum leaf extract. For example, the composition may comprise 100-3000 mg of sour cherry (Prunus cerasus). As a further example, the composition may comprise 25-100 mg Apocynum venetum leaf extract. In another embodiment of the present invention, the composition may also include between 10-100 mg cannabidiol (CBD). CBD is an active ingredient in cannabis that is derived from the hemp plant, but it does not contain tetrahydrocannabinol (THC), the psychoactive ingredient that produces a high. CBD is commonly used to treat chronic pain, inflammation, anxiety, and insomnia. It should also be clearly understood that substantial benefit may also be derived from the serving size being greater than or less than 1.23 ml. And it should also be understood that substantial benefit may also be derived from use of the composition in a solid form.

The foregoing description is provided to enable any person skilled in the relevant art to practice the various embodiments described herein. Various modifications to these embodiments will be readily apparent to those skilled in the relevant art, and generic principles defined herein can be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Moreover, nothing disclosed herein is intended to be dedicated to the public.

I claim:

1. A composition for improving sleep comprising:
an effective amount of tart cherry extract; and
an effective amount of Apocynum venetum leaf extract.

2. The composition of claim 1 wherein the effective amount of tart cherry extract is between 100 mg to 3000 mg.

3. The composition of claim 2 wherein the effective amount of tart cherry extract is 553 mg.

4. The composition of claim 1 wherein the effective amount of Apocynum venetum leaf extract is between 25 mg to 100 mg.

5. The composition of claim 4 wherein the effective amount of Apocynum venetum leaf extract is 25 mg.

6. The composition of claim 1 further comprising an effective amount of cannabidiol.

7. The composition of claim 6 wherein the effective amount of cannabidiol is between 10 mg to 100 mg.

8. The composition of claim 1 wherein the composition is in liquid form.

9. The composition of claim 8 wherein one serving size of the composition is equal to 1.23 ml.

10. A composition for improving sleep comprising:
between 100 mg to 3000 mg of Prunus cerasus extract; and
between 25 mg to 100 mg of Apocynum venetum leaf extract.

11. The composition of claim 10, comprising 553 mg of Prunus cerasus extract.

12. The composition of claim 10, comprising 25 mg of Apocynum venetum leaf extract.

13. The composition of claim 10 further comprising between 10 mg to 100 mg of cannabidiol.

14. The composition of claim 10 wherein the composition is in liquid form.

15. The composition of claim 14 wherein one serving size of the composition is equal to 1.23 ml.

16. A liquid composition for improving sleep comprising:
553 mg of Prunus cerasus extract;
25 mg of Apocynum venetum leaf extract; and
between 10 mg to 100 mg of cannabidiol.

17. The composition of claim 16 wherein one serving size of the composition is equal to 1.23 ml.
